(19) United States
(12) Patent Application Publication
(10) Pub. No.: US 2026/0263073 A1
(43) Pub. Date: Sep. 10, 2026

(54) SYSTEMS AND METHODS FOR A MANUALLY-ACTUATED FASTENER DELIVERY DEVICE

(71) Applicant: Dignity Health, San Francisco, CA (US)

(72) Inventors: Tyler Cole, San Francisco, CA (US); Dakota Graham, San Francisco, CA (US)

(21) Appl. No.: 18/993,544

(22) PCT Filed: Jul. 18, 2023
(86) PCT No.: PCT/US23/70443

§ 371 Date: Jan. 11, 2025

Related U.S. Application Data
(60) Provisional application No. 63/390,167, filed on Jul. 18, 2022.

(51) Int. Cl. A61B 17/11; A61B 17/00; A61B 17/30

(52) U.S. Cl. CPC A61B 17/11; A61B 2017/00946; A61B 2017/1107; A61B 2017/305

(57) ABSTRACT

A manually-actuated fastener delivery device for coupling portions of a bodily vessel or other cylindrical structure includes a first clamp portion that carries a first joinable portion of a fastener and a second clamp portion that carries a second joinable portion of the fastener. The fastener delivery device is optimized for micro-surgical applications, and aligns the respective first and second joinable portions of the fastener in a substantially parallel arrangement prior to coupling the first and second joinable portions of the fastener together.

### CROSS-REFERENCE TO RELATED APPLICATIONS

This is a PCT Patent Application that claims benefit to U.S. Provisional Patent Application Ser. No. 63/390,167 filed 18 Jul. 2022, which is herein incorporated by reference in its entirety.

### FIELD

The present disclosure generally relates to microsurgical devices, and in particular, to a system and associated method for a manually-actuated coupler delivery device for coupling vessels within superficial surgical sites or deep surgical corridors.

### BACKGROUND

Surgical fields that require microvascular anastomosis have seen many sutureless devices and techniques investigated for this purpose. Some of these devices and techniques include laser-assisted devices, staples, tissue adhesives, magnets, and couplers. Among microvascular surgeons in otolaryngology and reconstructive plastic surgery, the most studied microvascular anastomotic device is a “ring-and-pin” fastener. This device has the advantages of ease of use, quick anastomosis times, no intraluminal foreign material, and direct intima-to-intima contact. The fastener has been described as the preferred microvascular anastomotic technique for free tissue transfer at some institutions, and was initially applied to venous anastomoses but later found success in arterial anastomoses.

Although the fastener has been straightforwardly deployed in superficial end-to-end atrial bypasses, further technique development in deep tissue end-to-end coupled anastomosis is necessary for cerebrovascular use. Attempts to couple arteries in unreceptive fields like the Sylvian fissure cause cerebral cortical and vascular injury.

It is with these observations in mind, among others, that various aspects of the present disclosure were conceived and developed.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIG. 1A is an illustration showing a joinable part of a fastener for coupling two ends of a bodily lumen or other cylindrical structure body together;

FIG. 1B is an illustration demonstrating a problem with present fastener delivery devices;

FIGS. 2A and 2B are a pair of illustrations showing alignment of a first joinable part and a second joinable part of a fastener in a substantially parallel arrangement (FIG. 2A) and subsequent coupling of the first joinable part and the second joinable part of the fastener in an interlocking arrangement (FIG. 2B);

FIG. 3A is an illustration showing a fastener delivery device disclosed herein for coupling of the first joinable part and the second joinable part of the fastener of FIGS. 2A and 2B and having a clamp mechanism in a first position;

FIG. 3B is an illustration showing the fastener delivery device of FIG. 3A coupled with a mounting device;

FIG. 4 is an illustration showing an enlarged view of the clamp mechanism of the fastener delivery device of FIG. 3A in a second position corresponding to the substantially parallel arrangement of FIG. 2A:

FIGS. 5A-5C are a series of illustrations showing various perspective views of a first clamp portion of the clamp mechanism of FIG. 4;

FIG. 6 is an illustration showing an exploded view of the clamp mechanism of FIG. 4;

FIGS. 7A-7C are a series of illustrations showing a sequence for coupling the first joinable part and the second joinable part of the fastener by the fastener delivery device of FIG. 3A;

FIGS. 8A-8C are a series of illustrations showing various locking mechanisms of the fastener delivery device that may be used to “lock” the clamp mechanism in the second position of FIG. 4;

FIGS. 9A-9C are a series of side views of various alternative body styles of the fastener delivery device of FIG. 3A;

FIGS. 10A-10D are a series of front perspective views of various alternative path styles of the fastener delivery device of FIG. 3A;

FIGS. 11A and 11B are a pair of sequential perspective views of the fastener delivery device of FIG. 3A having a sleeve portion that assists in drawing portions of the clamp mechanism together for coupling the first joinable part and the second joinable part of the fastener;

FIGS. 12A-12D are a series of perspective and sequential enlarged views of an alternative embodiment of the fastener delivery device of FIG. 3A having a single hinge and a first receptacle and a second receptacle that can be drawn together in a sliding motion;

FIGS. 13A-13I are a series of photographs and illustrations showing coupling of two portions of a bodily lumen (or another cylindrical structure within the operative field) using the fastener delivery device of FIG. 3A in a surgical environment; and

FIG. 14 is a process flow chart showing an example method for coupling two portions of a bodily lumen or other cylindrical structure by the fastener delivery device of FIG. 1A, and 2A-13I.

Corresponding reference characters indicate corresponding elements among the view of the drawings. The headings used in the figures do not limit the scope of the claims.

### DETAILED DESCRIPTION

Microvascular anastomosis can be a versatile medical procedure for use in connecting vessels, and has been applied in both venous and arterial contexts. The need to perform these procedures primarily stems from the need to revascularize areas of the brain after complex cerebral aneurysms or resection of brain tumors. A substantial portion of microvascular anastomosis procedures performed involve either end-to-end anastomosis or end-to-side anastomosis.

End-to-end anastomosis involves connecting the ends of a vessel. The vessel is cut by the surgeon, exposing the ends of the vessel. There can either be one vessel or two vessels being connected, it depends on the specific problem that the surgeon is trying to address and is patient specific. The procedure traditionally uses sutures with the aid of a microscope. A newer method utilizes a fastener having first and second joinable parts to simplify the procedure and eliminate the need for suturing together the two ends. First, both ends of the vessel are inserted into their respective first or second joinable part. Next, the ends of the vessel are everted onto pins of the first and second joinable parts, such that the pins on the first and second joinable parts are facing one another. This also exposes the intima of each end to one another. Last, the first and second joinable parts are pushed together, thus coupling the ends of the vessel together, with intima-to-intima contact. The fastener comprised of the joinable parts is held together by pins inserted into respective pin holes of the first and second joinable parts, and no sutures are needed.

End-to-side anastomosis involves connecting the end of a donor vessel to a recipient vessel. This method also traditionally uses sutures, to suture the end of the donor vessel to a hole found on the recipient vessel. The combination can be thought of as a “T” shape, with the top of the “T” being the recipient vessel, and the vertical line being the donor vessel. The hole is placed on the recipient vessel by the surgeon, and the end is cut into a specific shape to fit onto the hole. The vessels being connected are dependent on the procedure and the specific problem of the patient. Fasteners have not been designed for end-to-side anastomosis, but these devices have been tested in end-to-side anastomosis. First, the end of the donor vessel is inserted and everted over the pins, like end-to-end anastomosis, with the intima exposed. Next, a hole on the recipient vessel which has excess wall of the vessel exposed such that the wall is inserted into the associated first or second joinable part and everted over the pins, also exposing the intima of the recipient vessel. Last, the first and second joinable parts are pushed together, thus coupling the donor vessel to the recipient vessel, with intima-to-intima contact, and without the need for sutures.

The present disclosure provides systems and methods for a manually-actuated microvascular anastomotic fastener delivery device for coupling vessels within superficial surgical sites or deep surgical corridors. In some embodiments, the device can be configured to access a vessel of a patient in a surgical corridor of approximately four centimeters below a tissue surface of the patient. In other embodiments, a device may be capable of entering a surgical corridor of more or less than four centimeters below the tissue surface. The device can be employed by any surgeon familiar with microvascular anastomosis.

### Fastener

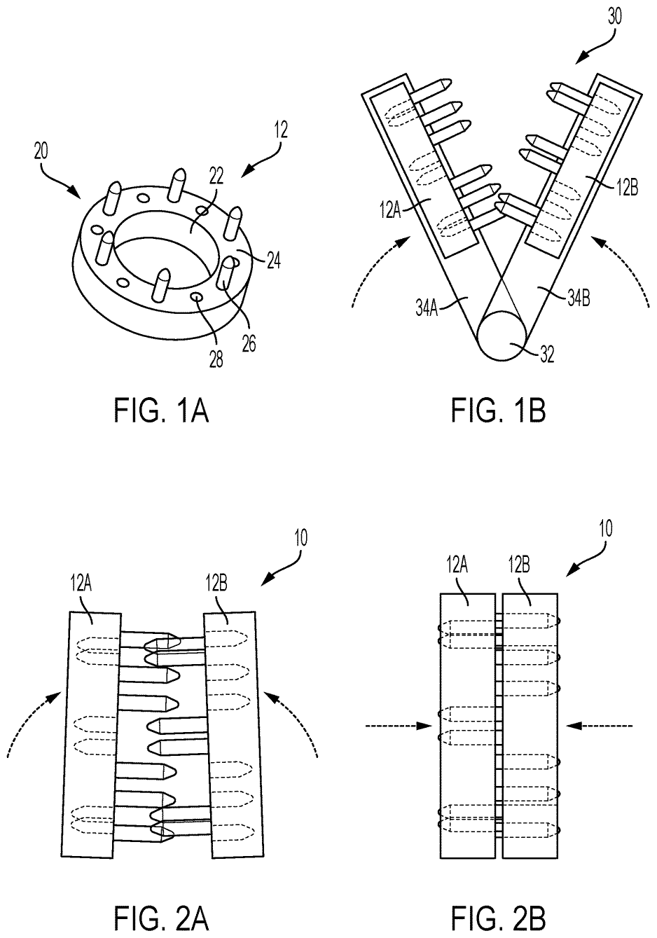

FIG. 1A shows a joinable part 12 of a fastener for coupling two ends of a bodily lumen or other cylindrical structure within the body together. As shown, the joinable part 12 includes a disc body 20 having a disc aperture 22. The joinable part 12 also defines an opposing face 24 configured for engagement with an opposing face of an additional joinable part (analogous to joinable part 12 shown in FIG. 1A). The opposing face 24 notably includes a plurality of pins 26 interposed between a plurality of receiving apertures 28. When the joinable part 12 is coupled with the additional joinable part, the plurality of pins 26 of the joinable part 12 insert into a plurality of receiving apertures of the additional joinable part; likewise, the plurality of receiving apertures 28 of the joinable part 12 receive the plurality of pins of the additional joinable part.

Currently, many fastener delivery devices such as a fastener delivery device 30 shown in FIG. 1B have a first clamp portion 34A and a second clamp portion 34B that pivot about a common hinge 32 to draw a first joinable part 12A and a second joinable part 12B. However, a problem arises when the plurality of pins of the first joinable part 12A do not properly align with the plurality of receiving apertures of the second joinable part 12B (and vice-versa). Some fastener delivery devices ensure alignment between respective joinable parts of a fastener by extending the distance from the joinable parts to a common hinge 32. From the perspective of the surgeon, this results in a relatively large footprint area that occupies useable operative space and is not ideal for small operative corridors.

As such, referring to FIGS. 2A and 2B, one aim of the present disclosure is to provide a fastener delivery device that enables alignment of the first joinable part 12A and the second joinable part 12B of a fastener 10 prior to coupling while minimizing the space occupied by the device. In FIG. 2A, the first joinable part 12A and the second joinable part 12B can first be oriented into a position where the first joinable part 12A and the second joinable part 12B are substantially parallel with one another. When the first joinable part 12A and the second joinable part 12B are substantially parallel with one another before coupling, it is important that each pin of the plurality of pins 26 (FIG. 1A) aligns with a respective receiving aperture of the plurality of receiving apertures 28 (FIG. 1A). Once aligned as shown in FIG. 2A, the first joinable part 12A and the second joinable part 12B can then be drawn together as shown in FIG. 2B to couple the first joinable part 12A and the second joinable part 12B together in an interlocking arrangement. This sequence of first orienting the first joinable part 12A and the second joinable part 12B such that they are substantially parallel with one another and then drawing the first joinable part 12A and the second joinable part 12B together provides practitioners with more control over the coupling of bodily lumens using the fastener 10.

### Fastener Delivery Device

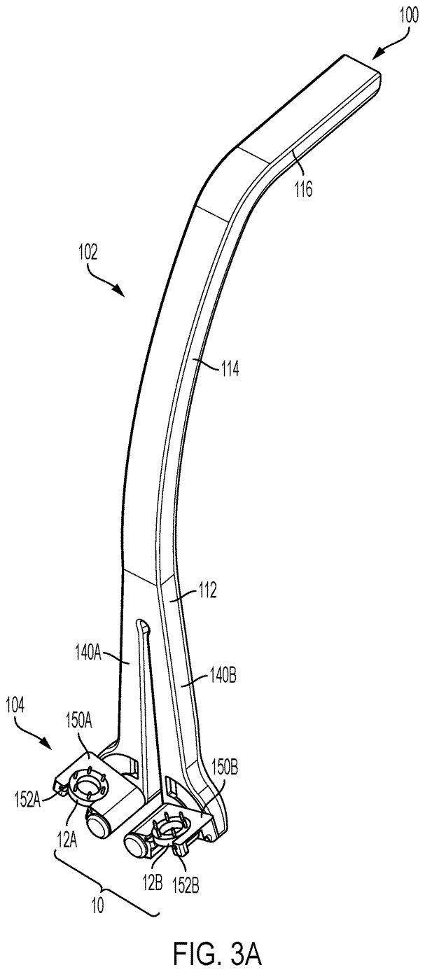

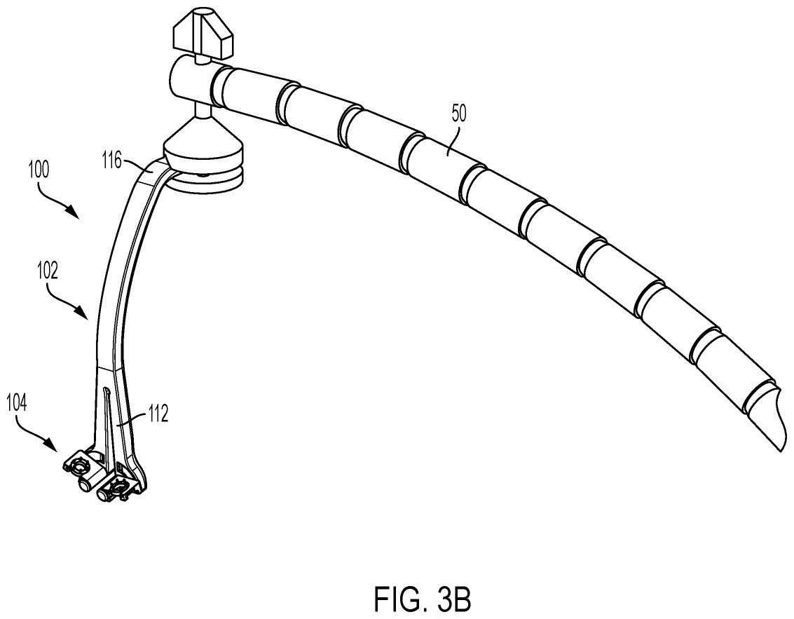

Referring to FIGS. 3A and 3B, a fastener delivery device 100 includes a body 102 having a distal portion 112 that defines a clamp mechanism 104 for coupling respective first and second joinable parts 12A and 12B of the fastener 10 together within an operative field. Importantly, the fastener delivery device 100 and other variations discussed herein aim to align the first and second joinable parts 12A and 12B of the fastener 10 with one another such that they are substantially parallel with one another (FIG. 2A) before coupling the first and second joinable parts 12A and 12B of the fastener 10 together in an interlocking arrangement (FIG. 2B).

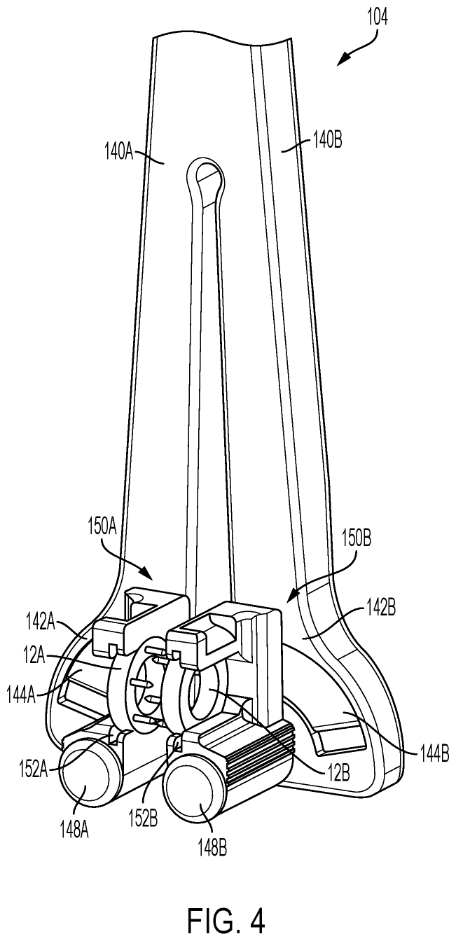

The clamp mechanism 104 includes a first clamp portion 150A that is pivotable between a first position (FIG. 3A) and a second position (FIG. 4) and includes a first receptacle 152A configured to receive the first joinable part 12A of the fastener 10. Likewise, the clamp mechanism 104 also includes a second clamp portion 150B that is pivotable between a first position (FIG. 3A) and a second position (FIG. 4) and includes a second receptacle 152B configured to receive the second joinable part 12B of the fastener 10.

The distal portion 112 of the body 102 can bifurcate into a first leg 140A and a second leg 140B as shown. In a primary embodiment, the first leg 140A and the second leg 140B are flexibly biased away from one another but can be manually drawn towards one another by a practitioner. The body 102 can further include an intermediate portion 114 and a proximal portion 116. As shown in the embodiment of FIG. 3A, the intermediate portion 114 can include a curve that points the proximal portion 116 away from the operative field to avoid obstructing a view of the practitioner. The proximal portion 116 can engage or include a mounting device 50 as shown in FIG. 3B; in the example of FIG. 3B, the mounting device 50 is a Greenburg mount, however, any other suitable mounting device can be used at the discretion of the practitioner. In some embodiments, the proximal portion 116 of the body 102 can form a mounting device (e.g., where the mounting device and the proximal portion 116 of the body 102 are not separable from one another). The proximal portion 116 can also be manually held by a practitioner.

### Clamp Mechanism

FIG. 4 shows an enlarged view of the clamp mechanism 104 of the fastener delivery device 100 with the first clamp portion 150A and the second clamp portion 150B in the second position. As shown, the first leg 140A includes a first face 142A defining a first path 144A that guides the first clamp portion 150A as the first clamp portion 150A pivots about a first hinge 148A between the first position of FIG. 3A and the second position of FIG. 4. Similarly, the second leg 140B includes a second face 142B defining a second path 144B that guides the second clamp portion 150B as the second clamp portion 150B pivots about a second hinge 148B between the first position of FIG. 3A and the second position of FIG. 4. When in the second position of FIG. 4, the first clamp portion 150A and the second clamp portion 150B are substantially parallel with one another.

As shown, the first receptacle 152A receives the first joinable part 12A of the fastener 10 and the second receptacle 152B receives the second joinable part 12B of the fastener 10 such the first joinable part 12A and the second joinable part 12B are substantially parallel with one another when the first clamp portion 150A and the second clamp portion 150B are in the second position. When in the second position, a first plurality of pins of the first joinable part 12A align with a second plurality of receiving apertures of the second joinable part 12B and a first plurality of receiving apertures of the first joinable part 12A align with a second plurality of pins of the second joinable part 12B.

When the first clamp portion 150A and the second clamp portion 150B are substantially parallel with one another as in the second position, the first clamp portion 150A and the second clamp portion 150B can then be manually drawn together by the practitioner to couple the first joinable part 12A with the second joinable part 12B in an interlocking arrangement. In contrast with previous fastener delivery devices, the fastener delivery device 100 of the present disclosure ensures that the first joinable part 12A and the second joinable part 12B are substantially parallel with one another before coupling. This can prevent complications that may arise when the pins and receiving apertures of the fastener 10 approach one another at an angle as shown in the example of FIG. 1B.

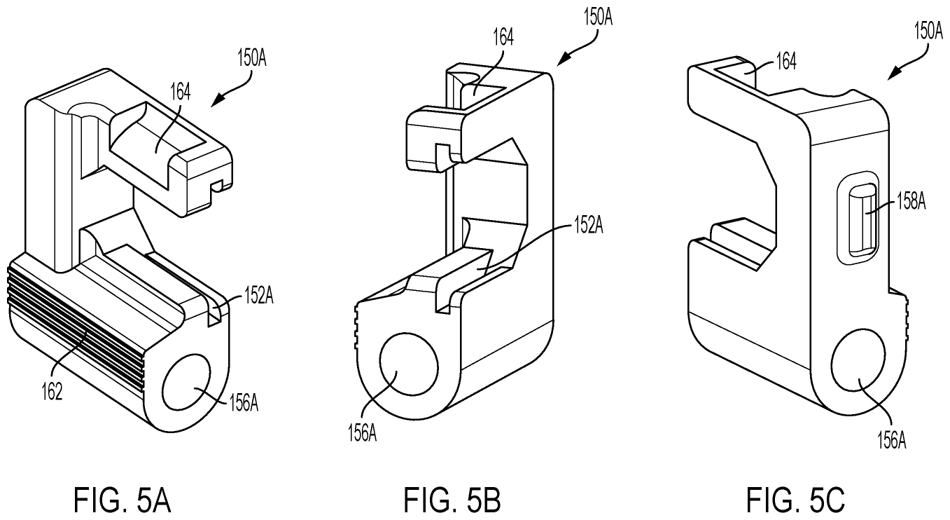

FIGS. 5A-5C show various perspective views of the first clamp portion 150A. The second clamp portion 150B shown in FIGS. 3A and 4 can be nearly identical to the first clamp portion 150A, but mirrored horizontally. Discussion with respect to components shown in FIGS. 5A-5C is provided here with respect to the first clamp portion 150A but is also applicable to the second clamp portion 150B. FIG. 5A shows a first side perspective view of the first clamp portion 150A, which includes the first receptacle 152A for receipt of the first joinable part 12A. As illustrated, the first clamp portion 150A includes a first hinge channel 156A that receives the first hinge 148A for engaging the first clamp portion 150A to the first face 142A of the clamp mechanism 104 (likewise, the second clamp portion 150B includes a second hinge channel that receives the second hinge 148B). FIG. 5A also shows A gripping surface 162 along an “external” side of the first clamp portion 150A, as well as A gripping notch 164 along a free end of the first clamp portion 150A. Gripping surface(s) 162 and gripping notch(es) 164 provide engagement points for the practitioner to rotate the first clamp portion 150A and the second clamp portion 150B between the first position of FIG. 3A and the second position of FIG. 4 and to draw the first clamp portion 150A and the second clamp portion 150B together using an instrument.

FIG. 5B shows an opposite side view of the first clamp portion 150A. As shown, an “internal” side of the first clamp portion 150A (or second clamp portion 150B) can be substantially smooth or flat to ensure proper contact with the second clamp portion 150B (or first clamp portion 150A) of FIG. 4 when coupling the first joinable part 12A with the second joinable part 12B in an arrangement described in greater detail herein with respect to FIGS. 5A-5C.

FIG. 5C shows a rear view of the first clamp portion 150A, which can include a first follower 158A that engages the first path 144A. As the first clamp portion 150A pivots between the first position of FIG. 3A and the second position of FIG. 4, the first follower 158A follows the first path 144A. Likewise, the second clamp portion 150B shown in FIGS. 3A and 4 includes a second follower (not visible) that similarly engages the second path 144B.

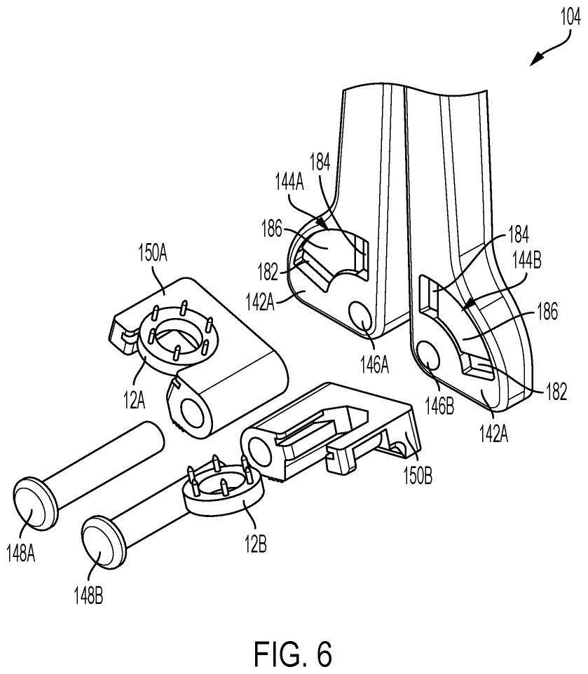

FIG. 6 shows an exploded view of the clamp mechanism 104 of the fastener delivery device 100. The first path 144A and the second path 144B each include a first friction element 182 associated with the first position of FIG. 3A and a second friction element 184 associated with the second position of FIG. 4. The first follower 158A (FIG. 5C) of the first clamp portion 150A engages the first friction element 182 of the first path 144A when in the first position and engages the second friction element 184 of the first path 144A when in the second position. Likewise, the second follower (not visible, but analogous to the first follower 158A) of the second clamp portion 150B engages the first friction element 182 of the second path 144B when in the first position and engages the second friction element 184 of the second path 144B when in the second position. As further shown, the first path 144A and the second path 144B can each include an intermediate section 186 that may include additional friction elements, and may otherwise define a recessed pathway that guides the first follower 158A of the first clamp portion 150A or the second follower of the second clamp portion 150B between the first position and the second position.

FIG. 6 also shows a first hinge receptacle 146A associated with the first face 142A of the first leg 140A and a second hinge receptacle 146B associated with the second face 142B of the second leg 140B. The first hinge receptacle 146A receives the first hinge 148A to connect the first clamp portion 150A with the first face 142A and the second hinge receptacle 146B receives the second hinge 148B to connect the second clamp portion 150B with the second face 142B. Further, the first joinable part 12A and the second joinable part 12B are respectively removeable from the first receptacle 152A of the first clamp portion 150A and the second receptacle 152B of the second clamp portion 150B.

### Coupling Sequence

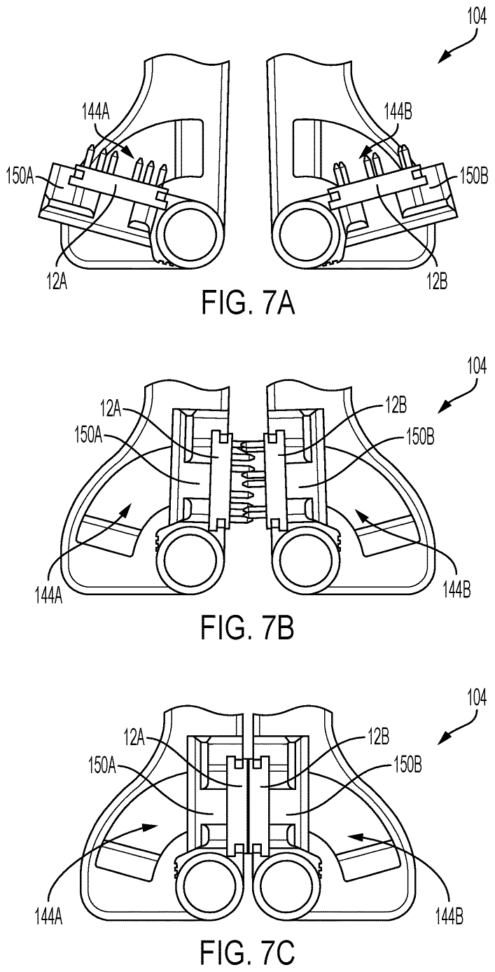

FIGS. 7A-7C show a sequence for coupling the first joinable part 12A and the second joinable part 12B of the fastener 10 using the fastener delivery device 100. FIG. 7A shows an enlarged front view of the clamp mechanism 104 of the fastener delivery device 100 where the first clamp portion 150A and the second clamp portion 150B are in the first position. In FIG. 7A, the first clamp portion 150A is shown engaging the first friction element 182 of the first path 144A and the second clamp portion 150B is shown engaging the first friction element 182 of the second path 144B. In the first position of FIG. 7A, bodily lumens or other compatible cylindrical structures can be inserted through the first joinable part 12A and the second joinable part 12B of the fastener 10 and everted onto pins of the first joinable part 12A and the second joinable part 12B.

FIG. 7B shows an enlarged front view of the clamp mechanism 104 of the fastener delivery device 100 where the first clamp portion 150A and the second clamp portion 150B have pivoted about respective first and second hinges 148A and 148B into the second position. As shown, the first clamp portion 150A and the second clamp portion 150B are substantially parallel to one another when in the second position, and the first joinable part 12A and the second joinable part 12B of the fastener 10 are also substantially parallel to one another when in the second position but are not yet coupled with one another. In the second position of FIG. 7B, respective ends of the bodily lumens are aligned with one another and the pins of the first joinable part 12A and the second joinable part 12B align with receiving apertures of first joinable part 12A and the second joinable part 12B. The second position of FIG. 7B enables a practitioner to ensure that the first joinable part 12A and the second joinable part 12B of the fastener are properly aligned before coupling.

FIG. 7C shows an enlarged front view of the clamp mechanism 104 of the fastener delivery device 100 where the first clamp portion 150A and the second clamp portion 150B have been manually drawn towards one another from the second position of FIG. 7C such that the first joinable part 12A engages the second joinable part 12B of the fastener 10 in the interlocking arrangement. In some examples, the first leg 140A and the second leg 140B are flexible enough such that they can be manually drawn towards one another, thereby drawing the first clamp portion 150A with the first joinable part 12A and the second clamp portion 150B with the second joinable part 12B together. Further, the gripping surfaces 162 and gripping notches 164 of the first clamp portion 150A and the second clamp portion 150B can provide engagement points for the practitioner to use an instrument to draw the first clamp portion 150A and the second clamp portion 150B together. Once the first joinable part 12A and the second joinable part 12B are coupled together, the fastener 10 (and associated bodily lumens or other compatible cylindrical structure within the operative field) can be decoupled from the first receptacle 152A of the first clamp portion 150A and the second receptacle 152B of the second clamp portion 150B.

### Locking Mechanisms

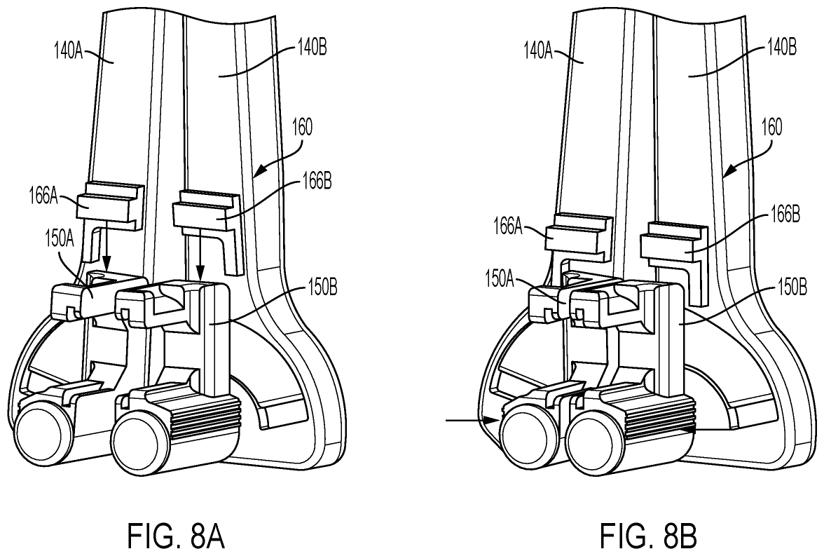

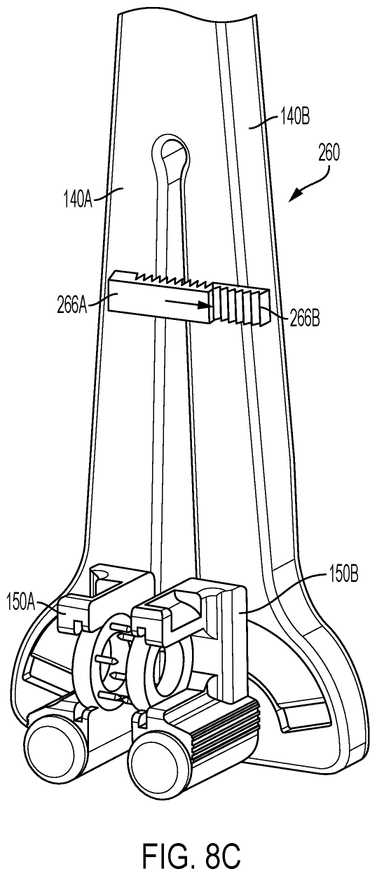

In some embodiments shown in FIGS. 8A and 8B, the clamp mechanism 104 of the fastener delivery device 100 can include a locking mechanism 160 that can be used to secure the first clamp portion 150A and the second clamp portion 150B in the second position of FIGS. 2A and 7B prior to coupling. As illustrated, the locking mechanism 160 can include a first lock 166A that engages the first clamp portion 150A and a second lock 166B that engages the second clamp portion 150B. The first lock 166A is moveable in a substantially vertical direction along the first leg 140A between a disengaged position shown in FIG. 8A and an engaged position shown in FIG. 8B. In the engaged position, the first lock 166A captures a free end of the first clamp portion 150A when the first clamp portion 150A is in the second position. Similarly, the second lock 166B is moveable in a substantially vertical direction along the second leg 140B between a disengaged position shown in FIG. 8A and an engaged position shown in FIG. 8B. In the engaged position, the second lock 166B captures a free end of the second clamp portion 150B when the second clamp portion 150B is in the second position.

Following engagement of the locking mechanism 160 as shown in FIG. 8B, the first clamp portion 150A and the second clamp portion 150B can be manually drawn together to couple the first joinable part 12A and the second joinable part 12B of the fastener together. The locking mechanism 160 prevents the first clamp portion 150A and the second clamp portion 150B from falling out of the substantially parallel arrangement of the second position.

FIG. 8C shows an alternative locking mechanism 260 including a first lock 266A associated with the first leg 140A and a second lock 266B associated with the second leg 140B. The first lock 266A and the second lock 266B can include a plurality of ridges interposed with a plurality of recesses that incrementally engage one another as the first leg and the second leg are drawn together such as in FIG. 7C and prevent the first clamp portion 150A and the second clamp portion 150B from falling out of the substantially parallel arrangement of the second position.

### Alternative Body Styles

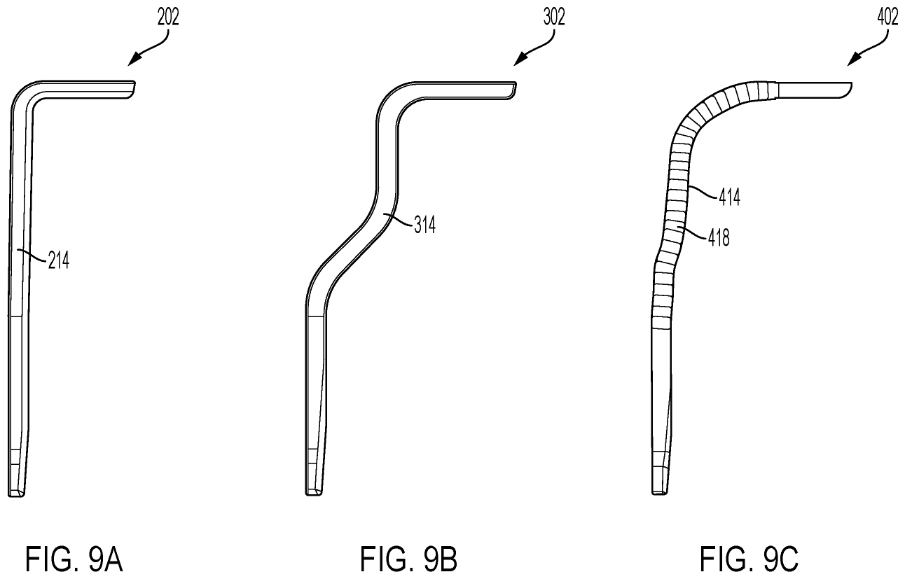

FIGS. 9A-9C show side views of various alternative styles of the body 102 shown in FIG. 3A. While the intermediate portion 114 of the body 102 shown in FIG. 3A includes a curve, an intermediate portion 214 of a body 202 (e.g., of a fastener delivery device) can be straight as shown in FIG. 9A. FIG. 9B shows another intermediate portion 314 of a body 302 (e.g., of a fastener delivery device) that includes at least two curves, e.g., to resemble a “bayonet”.

In some examples, the body of the fastener delivery device can be malleable and memory-retaining (e.g., of a softer metallic material that is manually deformable or may be morphed to predetermined shape configurations and may temporarily retain such configurations) so that the practitioner can shape the body of the fastener delivery device as needed for the operative space. FIG. 9C shows yet another intermediate portion 414 of a body 402 (e.g., of a fastener delivery device) that includes a plurality of body segments 418 that can be loosened, adjusted, and/or tightened to allow the body 402 to articulate as needed.

### Alternative Path Styles

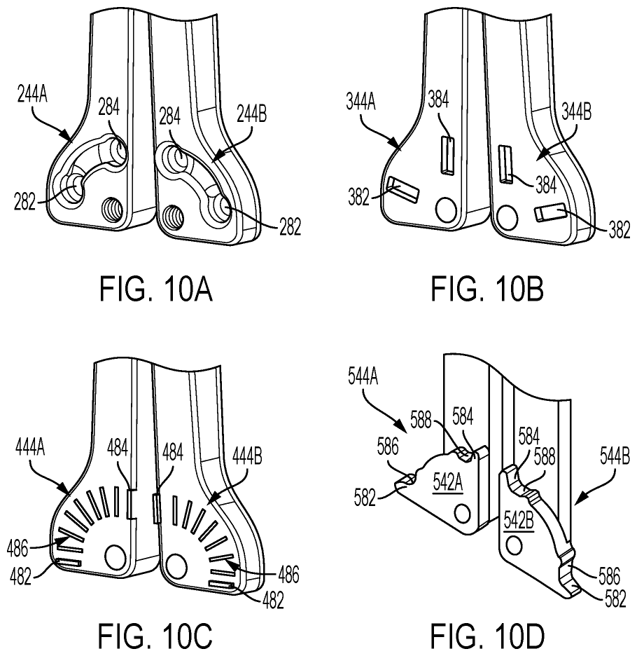

FIGS. 10A-10D show front perspective views of alternative path styles of the first and second paths 144A and 144B shown in FIG. 6. While the first and second paths 144A and 144B shown in FIG. 6 include first and second friction elements 182 and 184 that are rectangular in shape and are recessed into the first and second paths 144A and 144B, FIG. 10A shows first and second paths 244A and 244B having first and second friction elements 282 and 284 that are rounded in shape.

Further, while the first and second paths 144A and 144B shown in FIG. 6 each include intermediate sections 186, FIG. 10B shows first and second paths 344A and 344B each having first and second friction elements 382 and 384 that are recessed into the first and second paths 344A and 344B but lack an intermediate section.

In another embodiment, while FIG. 6 shows first and second friction elements 182 and 184 that are recessed into the first and second paths 144A and 144B, FIG. 10C shows first and second paths 444A and 444B having first and second friction elements 482 and 484 along with a plurality of intermediate friction elements 486 that are raised rather than recessed. The first and second friction elements 482 and 484 can act as stoppers, and can extend farther outward than the plurality of intermediate friction elements 486 keeping a first clamp portion (e.g., first clamp portion 150A) and a second clamp portion (e.g., second clamp portion 150B) along the first and second paths 444A and 444B.

FIG. 10D shows another alternative arrangement in which first and second paths 544A and 544B are positioned along respective top surfaces of first and second faces 542A and 542B as shown, with first and second friction elements 582 and 584 similarly acting as stoppers, keeping a first clamp portion (e.g., first clamp portion 150A) and a second clamp portion (e.g., second clamp portion 150B) along the first and second paths 544A and 544B. In some examples, the first and second paths 544A and 544B each define a first seat 586 and a second seat 588 that receive followers of the first clamp portion and second clamp portion and maintain the first clamp portion and second clamp portion in the first position or the second position.

### Sleeve Portion

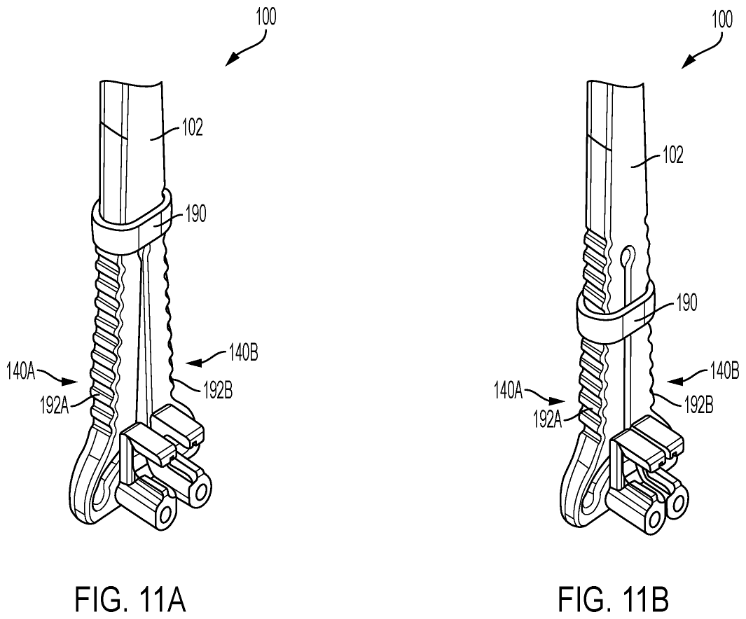

Referring to FIGS. 11A and 11B, in some embodiments the fastener delivery device 100 can include a sleeve portion 190 that is moveable along the body 102 that enables the practitioner to draw the first clamp portion 150A and the second clamp portion 150B (and thereby, the first joinable part 12A and the second joinable part 12B of FIGS. 2A and 2B) together by advancing the sleeve portion 190 towards the distal portion 112 of the body 102. FIG. 11A in particular shows the fastener delivery device 100 in the second position. In FIG. 11A, the sleeve portion 190 is positioned along the body 102 proximal to where the bifurcation forming the first leg 140A and the second leg 140B starts. FIG. 11B shows the sleeve portion 190 positioned along the body 102 distal to where the bifurcation forming the first leg 140A and the second leg 140B starts. As the sleeve portion 190 advances down the body 102, the first leg 140A and the second leg 140B are drawn together, and consequently, the first clamp portion 150A and the second clamp portion 150B are drawn together. In some embodiments, the first leg 140A and the second leg 140B respectively include friction surfaces 192A and 192B along external portions of the first leg 140A and the second leg 140B that provide friction for the sleeve portion 190, preventing the sleeve portion from unintentionally sliding back upwards towards a proximal portion of the body 102.

### Alternative Clamp Mechanism

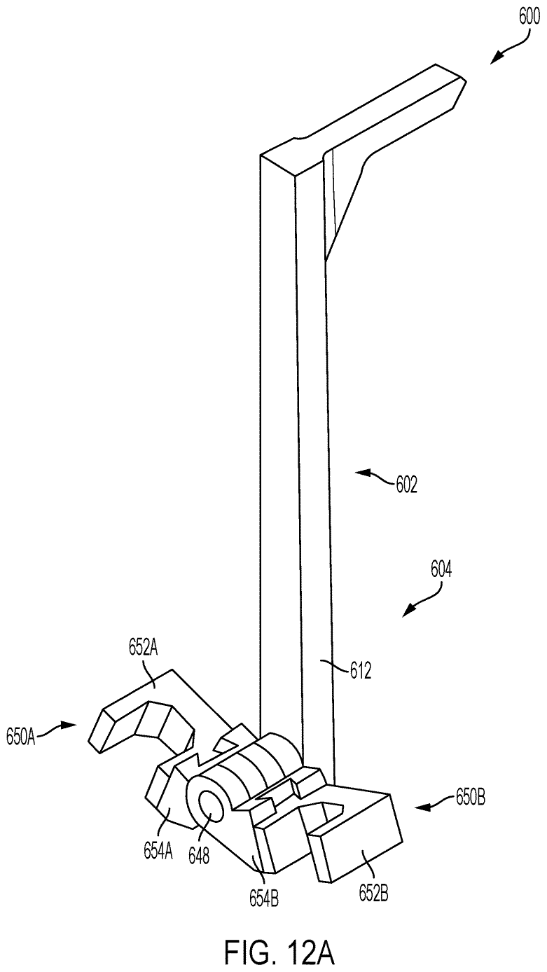

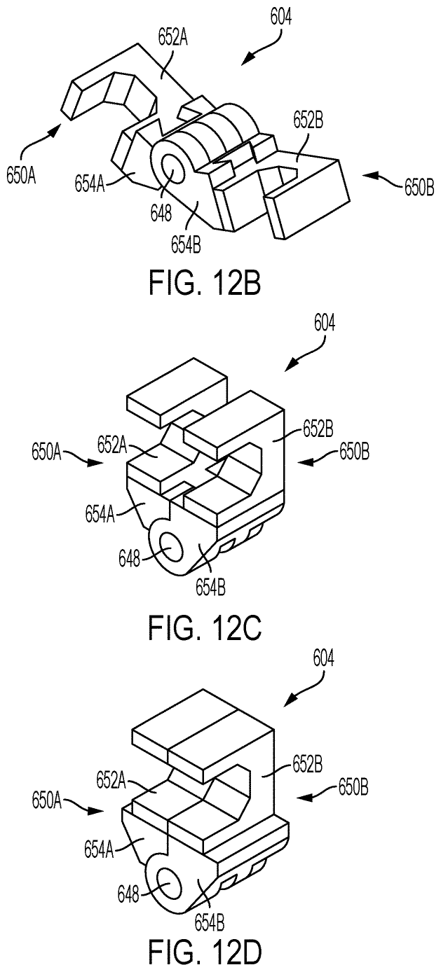

FIGS. 12A-12D show a fastener delivery device 600 having a clamp mechanism 604 at a distal portion 612 of a body 602. The clamp mechanism 604 includes a first clamp portion 650A and a second clamp portion 650B that are each pivotable between a first position (FIG. 12B) and a second position (FIG. 12C). The first clamp portion 650A and the second clamp portion 650B are substantially parallel with one another when in the second position. The first clamp portion 650A and the second clamp portion 650B are similarly configured to be drawn towards one another in a sliding motion as shown in FIG. 12D when the first clamp portion 650A and the second clamp portion 650B are substantially parallel with one another.

As illustrated, the first clamp portion 650A can include a first base 654A that is pivotable about a hinge 648 between the first position of FIG. 12B and the second position of FIG. 12C. Likewise, the second clamp portion 650B can include a second base 654B that is pivotable about the hinge 648 between the first position of FIG. 12B and the second position of FIG. 12C. As shown, the hinge 648 extends from the distal portion 612 of the body in a direction that is perpendicular to a direction of elongation of the body 602.

The first clamp portion 650A can also include a first receptacle 652A that is slidably mounted along the first base 654A and the second clamp portion 650B can similarly include a second receptacle 652B that is that is slidably mounted along the second base 654B, where the first receptacle 652A and the second receptacle 652B can be moved along a lateral direction as shown in FIGS. 12C and 12D. Similar to previous embodiments discussed herein, the first receptacle 652A and the second receptacle 652B can receive respective first and second joinable parts 12A and 12B of the fastener 10 (FIGS. 2A and 2B). When the first base 654A and the second base 654B are in the second position of FIG. 12C, the first joinable part 12A and the second joinable part 12B of the fastener 10 are substantially parallel with one another (such as in the arrangement of FIG. 2A). The first receptacle 652A and the second receptacle 652B can then be drawn together as shown in FIG. 12D to bring the first joinable part 12A and the second joinable part 12B of the fastener 10 together in an interlocking arrangement (such as in the arrangement of FIG. 2B).

In the example shown in FIGS. 12A-12D the fastener delivery device 600 is shown without bifurcation of the distal portion 612 of the body 602, in direct contrast with previously-discussed embodiments. This arrangement enables the fastener delivery device 600 to have a small footprint within the operative field while still enabling a practitioner to ensure that the first joinable part 12A and the second joinable part 12B of the fastener 10 are substantially parallel with one another before coupling. Further, while the example shown in FIG. 12A shows the body 602 having a “straight” profile similar to the example of FIG. 7A, the body 602 could also include one or more curves or can be malleable/adjustable such as in the examples of FIGS. 3A, 7B and 7C.

### Surgical Example

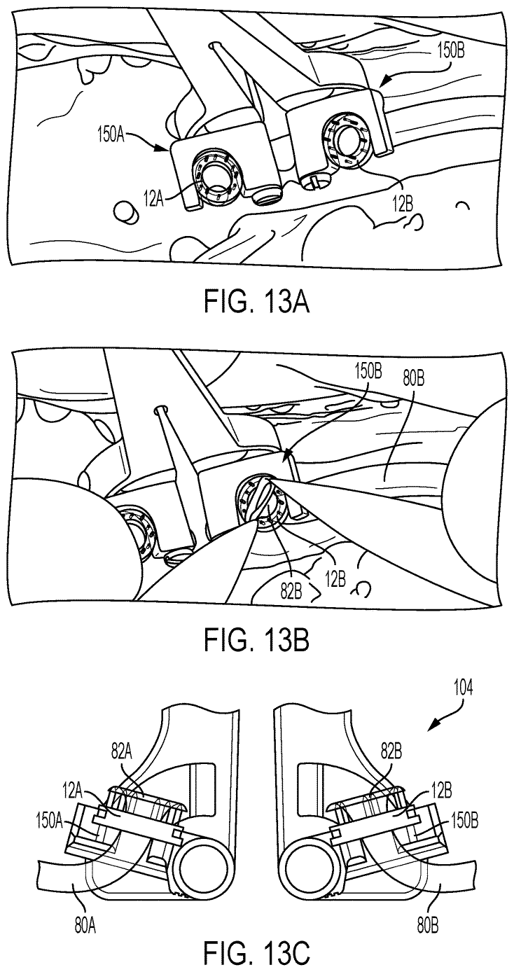

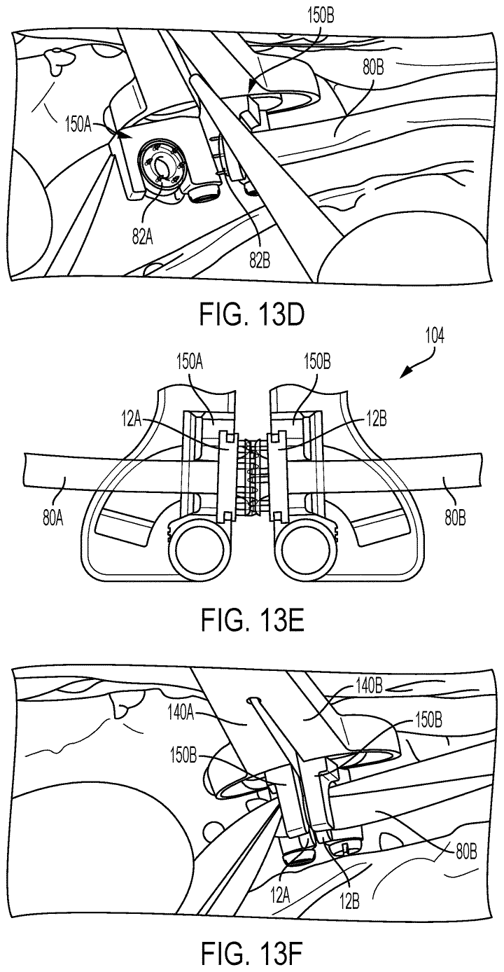

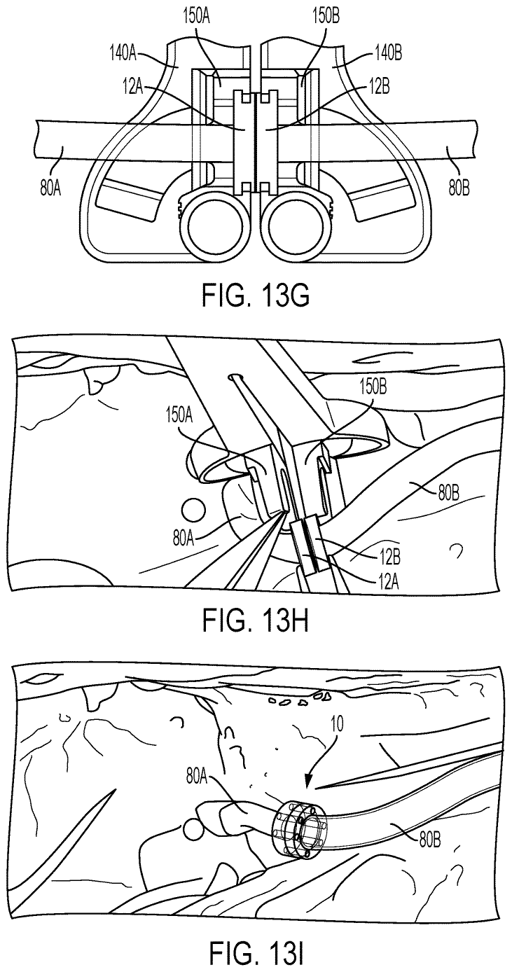

FIGS. 13A-13I are a series of images showing coupling of two portions of a bodily lumen (or another cylindrical structure within the operative field) using the fastener delivery device 100 (e.g., a first bodily lumen 80A and a second bodily lumen 80B). While the example shown is in terms of a microvascular surgical operation where the bodily lumen is a blood vessel, the methods and devices discussed herein can be similarly applied for coupling other bodily lumens such as those involved in the lymphatic system, digestive system, or other bodily systems. In a further aspect, the methods and devices discussed herein can be similarly applied for coupling cylindrical structures within the body including nerves.

As shown in FIG. 13A, the fastener delivery device 100 and fastener 10 is first placed within the operative field. In some embodiments, the first joinable part 12A of the fastener 10 can be pre-packaged in removeable engagement with the first clamp portion 150A and the second joinable part 12B of the fastener 10 can be pre-packaged in removeable engagement with the second clamp portion 150B. In FIGS. 13B and 13C, a first bodily lumen 80A is communicated through a disc aperture (FIG. 1A) of the first joinable part 12A of the fastener 10 and a second bodily lumen 80B is communicated through a disc aperture of the second joinable part 12B of the fastener 10. The first bodily lumen 80A is everted onto the pins (FIG. 1A) of the first joinable part 12A and the second bodily lumen 80B is everted onto the pins of the second joinable part 12B as shown. FIG. 13C shows a first everted portion 82A of the first bodily lumen 80A engaged with the first joinable part 12A of the fastener 10 and a second everted portion 82B of the second bodily lumen 80B engaged with the second joinable part 12B of the fastener 10, where the clamp mechanism 104 of the fastener delivery device 100 is in the first position.

FIGS. 13D and 13E show transitioning the clamp mechanism 104 of the fastener delivery device 100 to the second position following eversion of the first everted portion 82A and the second everted portion 82B onto the pins of the first joinable part 12A and the second joinable part 12B of the fastener 10. In particular, FIG. 13D shows pivoting the first clamp portion 150A and the second clamp portion 150B such that the first clamp portion 150A and the second clamp portion 150B are substantially parallel with one another. FIG. 13E shows the first clamp portion 150A and the second clamp portion 150B, and consequently the first joinable part 12A and the second joinable part 12B, in the second position in which they are substantially parallel with one another.

FIGS. 13F and 13G show closure or coupling of the first joinable part 12A and the second joinable part 12B of the fastener 10 by the clamp mechanism 104 of the fastener delivery device 100. In FIG. 13F, the first clamp portion 150A and the second clamp portion 150B are drawn towards one another such that the first joinable part 12A engages the second joinable part 12B in an interlocking arrangement. FIG. 13G shows the first joinable part 12A coupled with the second joinable part 12B in an interlocking engagement, while still engaged with the fastener delivery device 100. In this example, the first leg 140A and the second leg 140B are also shown being drawn towards one another.

FIG. 13H shows decoupling the first clamp portion 150A from the first joinable part 12A and decoupling the second clamp portion 150B from the second joinable part 12B. As shown, the first joinable part 12A and the second joinable part 12B of the fastener 10 can be removed through open portions of respective first and second receptacles 152A and 152B (FIG. 4) of the first clamp portion 150A and the second clamp portion 150B. Following decoupling of the first joinable part 12A and the second joinable part 12B from the fastener delivery device 100, the fastener delivery device 100 can be removed from the operative field.

FIG. 13I shows the fastener 10 remaining within the operative field, with first and second bodily lumens 80A and 80B being connected with one another by the fastener 10.

### Materials and Methods

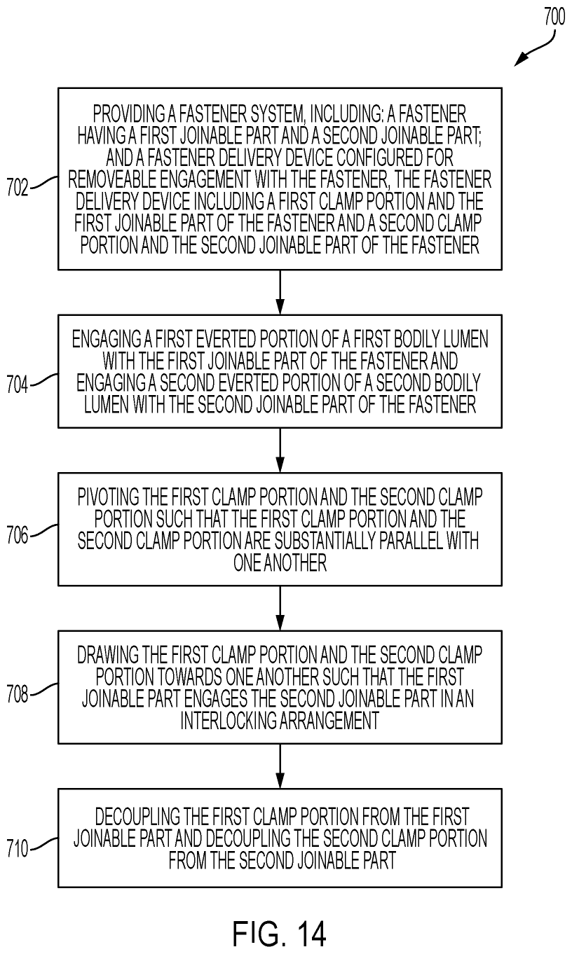

FIG. 14 shows a method 700 for coupling two portions of a bodily lumen or other cylindrical structure (such as a nerve). The method 700 can be applied using the fastener delivery device 100 and any variations thereof discussed herein with respect to FIG. 1A, and 2A-13I.

Step 702 of method 700 includes providing a fastener system, including: a fastener having a first joinable part and a second joinable part; and a fastener delivery device configured for removeable engagement with the fastener, the fastener delivery device including a first clamp portion and the first joinable part of the fastener and a second clamp portion and the second joinable part of the fastener. In some embodiments, the fastener may be pre-packaged with the fastener delivery device in a sterile package, where the first joinable part of the fastener is pre-loaded within the first clamp portion and the second joinable part of the fastener is pre-loaded within the second clamp portion.

The fastener delivery device can be made using manufacturing methods that are suitable for medical use, such as additive (e.g., 3-D printing) or reductive (e.g., milling) methods, molding (e.g., injection molding, compression molding, etc.), or casting (e.g., polymer solution casting, die casting, etc.) using biocompatible plastic or metal materials. In embodiments where the fastener is pre-packaged with the fastener delivery device in a sterile package, the fastener delivery device can be single-use. For a disposable embodiment, the fastener delivery device can include materials such as a biocompatible polymer, resin, or injection molded thermoplastic. The body of the fastener delivery device can be of a substantially rigid material, or can be of a malleable material or can be segmented such that the fastener delivery device can be selectively re-shaped as needed. Possible biocompatible materials include polyvinylchloride, polyethersulfone, polytetrafluoroethylene, polyethylene, polyurethane, polyetherimide, polycarbonate, polysulfone, polyetheretherketone, polypropylene, acrylonitrile butadiene styrene, polyamide, polystyrene, and polylactic acid. For a disposable embodiment, the fastener delivery device can be sterilized via ethylene oxide, gamma radiation, or a combination of both. In other embodiments, the fastener delivery device may be made of durable materials that can be thoroughly sterilized and re-used. For a reusable embodiment, the fastener delivery device can include a metallic material, such as stainless steel, substantially steel alloys, surgical grade steel, substantially titanium alloys, and substantially aluminum alloys. For a reusable embodiment, the fastener delivery device can be sterilized via autoclave.

In examples such as FIG. 9C where the body of the fastener delivery device is shapeable, step 702 can further include shaping the intermediate portion of the body of the fastener delivery device prior to introduction of the fastener delivery device into an operative field. In examples such as FIG. 3B that incorporate a mounting device, step 702 can further include mounting the fastener delivery device to a mounting device. Step 702 may also include introducing the fastener delivery device into the operative field as illustrated in FIG. 13A.

Step 704 of method 700 includes engaging a first everted portion of a first bodily lumen with the first joinable part of the fastener and engaging a second everted portion of a second bodily lumen with the second joinable part of the fastener, illustrated in FIGS. 13B and 13C. As discussed herein and as shown in FIG. 1A, the first joinable part and the second joinable part of the fastener each include a disc body defining a disc aperture and an opposing face having a plurality of pins interposed between a plurality of receiving apertures. As such, step 704 can further include sub-steps of: communicating a first bodily lumen through the disc aperture of the first joinable part and communicating a second bodily lumen through the disc aperture of the second joinable part; and everting the first bodily lumen onto the plurality of pins along the opposing face of the first joinable part and everting the second bodily lumen onto the plurality of pins along the opposing face of the second joinable part.

Step 706 of method 700 includes pivoting the first clamp portion and the second clamp portion such that the first clamp portion and the second clamp portion are substantially parallel with one another (e.g., transitioning the fastener delivery device between the first position shown in FIGS. 3A, 7A, and 13C and the second position shown in FIGS. 4, 7B, and 13E). In examples such as those in FIGS. 3A-11B that include first and second legs, step 706 can include pivoting the first clamp portion along the first face of the first leg to the second position and the second clamp portion along the second face of the second leg to the second position such that the first clamp portion and the second clamp portion are substantially parallel with one another.

In other examples, such as those in FIGS. 12A-12D where the first clamp portion includes a first base and a first receptacle slidably mounted along the first base and configured to receive the first joinable part, where the second clamp portion includes a second base and a second receptacle slidably mounted along the second base and configured to receive the second joinable part, and where the first and second bases are both pivotable about the hinge between the first position and the second position, step 706 can include pivoting the first base and the second base about the hinge to the second position such that the first receptacle and the second receptacle are substantially parallel with one another.

In some examples involving a locking mechanism such as those shown in FIGS. 8A-8C, step 706 can be followed with engaging the locking mechanism when the first clamp portion and the second clamp portion are substantially parallel.

Step 708 of method 700 includes drawing the first clamp portion and the second clamp portion towards one another such that the first joinable part engages the second joinable part in an interlocking arrangement. This step is illustrated in FIGS. 7C, 9B, 13F and 13G. In examples such as those in FIGS. 3A-11B that include first and second legs, step 708 can further include drawing the first leg and the second leg towards one another such that the first joinable part engages the second joinable part in the interlocking arrangement. In other examples, such as those in FIGS. 12A-12D (in particular, FIG. 12D), step 708 can further include sliding the first receptacle and the second receptacle towards one another such that the first joinable part engages the second joinable part in the interlocking arrangement.

Step 710 of method 700 includes decoupling the first clamp portion from the first joinable part and decoupling the second clamp portion from the second joinable part, and is illustrated in FIGS. 13H and 13I. Following step 710, the fastener delivery device can be removed from the operative field.

The functions performed in the processes and methods may be implemented in differing order. Furthermore, the outlined steps and operations are provided as examples, and some of the steps and operations may be optional, combined into fewer steps and operations, or expanded into additional steps and operations without detracting from the essence of the disclosed embodiments.

Some embodiments of the fastener delivery device can be used by a single surgeon. The surgeon can use both hands when placing the ends of the bodily lumen or other cylindrical structure into their respective first and second joinable parts of the fastener. When closing the fastener together, the surgeon might only need one hand to reconfigure the fastener delivery device, as the fastener delivery device can be mounted within the surgical space.

Further, the fastener delivery device and any variations thereof discussed herein with respect to FIG. 1A, and 2A-13I may be applied to and in conjunction with any number of conditions and surgical procedures, and the invention is in no way limited only to neurovascular use. The fastener delivery device can be applied for treatment of various diseases and/or conditions that require coupling or resection of bodily lumens (e.g., tubes such as blood vessels, lymphatic vessels, ducts, bowels, connections between organs or organ systems, etc.) or other cylindrical or non-tubular structures (e.g., nerves) within the body. The fastener delivery device can be used in any number of surgical subspecialties that perform microvascular anastomosis, such as and in no way limited to, cosmetic and/or reconstructive surgery, head and neck surgery, or other surgical applications that involve connection of two vessels such as bowel surgery or cardiovascular surgery. Examples include, but are not limited to: congenital diseases, cancers or other illnesses requiring resection and/or reconstruction of a bodily structure including associated tubular structures, aneurysms of blood vessels within the body, and physical injury requiring reconstruction of biological vessels and/or nerves.

As further described herein, various embodiments of devices and related methods herein utilize mechanisms and techniques that allow targeting of deep surgical corridors. In one embodiment, the fastener delivery device embodies a fastener delivery system that allows a user to close or connect a bodily lumen or other cylindrical structure at about a 90-degree angle, providing the user the capacity to safely target deep surgical corridors in the surgical procedure. As readily apparent to one of skill in the art, while previous designs related to ring-shaped fasteners having pins may be known in the art, various embodiments herein comprise one or more novel features of a device and related methods that allow the targeting of deep surgical corridors, e.g., by providing a fastener delivery device that aligns respective joinable portions of a fastener in a substantially parallel arrangement prior to coupling, while also maintaining a minimized “footprint” within the operative space. Many devices known in the art with pin and/or ring features are limited to use in broad or open corridors, which in turn, limit their utility and effectiveness.

While various examples of specific conditions or diseases are provided, it is appreciated that the tools and sequences outlined herein are not limited to these specific listed conditions and diseases, as readily apparent to one of skill in the art. Further, while there have been shown and described illustrative embodiments of a fastener delivery device showing specific sequences, tools, and views, it is to be understood that various other adaptations and modifications may be made within the spirit and scope of the embodiments herein. For example, the various embodiments have been shown and described herein as having specific features, however such features may be interchangeably included (or excluded) from any of the embodiments disclosed herein. It is also appreciated that the various discrete components or component parts of structures described herein can readily be incorporated or integrated into a larger structure. It is also appreciated that while some embodiments include a plurality or pairs of components, it is appreciated that a single component can be used with departing from the spirit and scope of this disclosure.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A fastener delivery device, comprising:
a first clamp portion being pivotable between a first position and a second position; and
a second clamp portion being pivotable between a first position and a second position;
wherein the first clamp portion and the second clamp portion are substantially parallel with one another when in the second position; and
wherein the first clamp portion and the second clamp portion are configured to be drawn towards one another when the first clamp portion and the second clamp portion are substantially parallel with one another.

2. The fastener delivery device of claim 1, wherein the first clamp portion includes a first receptacle configured to receive a first joinable part of a fastener and wherein the second clamp portion includes a second receptacle configured to receive a second joinable part of the fastener.

3. The fastener delivery device of claim 2, wherein the first joinable part and the second joinable part of the fastener are substantially parallel with one another when the first clamp portion and the second clamp portion are in the second position and wherein the first joinable part and the second joinable part of the fastener engage one another in an interlocking arrangement when the first clamp portion and the second clamp portion are drawn towards one another.

4. The fastener delivery device of claim 2, further comprising:
a first base of the first clamp portion, the first base being pivotable about a hinge between the first position and the second position, wherein the first receptacle is slidably mounted along the first base;
a second base of the second clamp portion, the second base being pivotable about the hinge between the first position and the second position, wherein the second receptacle is slidably mounted along the second base; and
a body having a distal portion, where the hinge extends from the distal portion of the body perpendicular to a direction of elongation of the body.

5. The fastener delivery device of claim 4, wherein the first joinable part and the second joinable part of the fastener are substantially parallel with one another when the first base and the second base are in the second position and wherein the first joinable part and the second joinable part of the fastener engage one another in an interlocking arrangement when the first receptacle and the second receptacle of the fastener delivery device are drawn towards one another.

6. The fastener delivery device of claim 1, wherein the first clamp portion and the second clamp portion each include one or more gripping surfaces.

7. The fastener delivery device of claim 1, further comprising:
a first leg having a first face in operative association with the first clamp portion, the first clamp portion being pivotable between the first position and the second position along the first face; and
a second leg having a second face in operative association with the second clamp portion, the second clamp portion being pivotable between the first position and the second position along the second face;
wherein drawing the first leg and the second leg towards one another when the first clamp portion and the second clamp portion are substantially parallel with one another results in the first clamp portion and the second clamp portion being drawn towards one another.

8. The fastener delivery device of claim 7, wherein the first leg and the second leg are bifurcations of a distal portion of a body.

9. The fastener delivery device of claim 7, the first leg and the second leg being flexibly biased away from one another.

10. The fastener delivery device of claim 7, wherein the first clamp portion is pivotable about a first hinge extending from the first face and wherein the second clamp portion is pivotable about a second hinge extending from the second face.

11. The fastener delivery device of claim 7, including:
a first follower associated with a free end of the first clamp portion, the first follower being in operative association with a first path along the first face; and
a second follower associated with a free end of the second clamp portion, the second follower being in operative association with a second path along the second face;
wherein the first path and the second path are mirrored relative to one another and wherein the first path and the second path respectively guide the free end of the first clamp portion and the free end of the second clamp portion between the first position and the second position.

12. The fastener delivery device of claim 11, wherein the first path and the second path each include a first friction element associated with the first position and a second friction element associated with the second position.

13. The fastener delivery device of claim 7, further comprising a sleeve portion and the first leg and the second leg each including a friction surface, the sleeve portion being moveable along the friction surfaces of the first leg and the second leg such that positioning the sleeve portion at a distal position along the friction surfaces of the first leg and the second leg draws the first leg and the second leg towards one another.

14. The fastener delivery device of claim 1, including:
a locking mechanism in operative association with the first clamp portion and the second clamp portion and configured to secure the first clamp portion and the second clamp portion in the second position.

15. The fastener delivery device of claim 14, the locking mechanism including:
a first lock along a first leg of the fastener delivery device and moveable between a disengaged position and an engaged position, wherein the first lock captures a free end of the first clamp portion when the first clamp portion is in the second position and when the first lock is in the engaged position; and
a second lock along a second leg of the fastener delivery device and moveable between a disengaged position and an engaged position, wherein the second lock captures a free end of the second clamp portion when the second clamp portion is in the second position and when the second lock is in the engaged position.

16. The fastener delivery device of claim 1, further comprising a body having an intermediate portion and defining one or more curves.

17. The fastener delivery device of claim 1, further comprising a body having an intermediate portion that is re-shapeable.

18. The fastener delivery device of claim 1, further comprising a body having a proximal portion configured for engagement with a mounting device.

19. The fastener delivery device of claim 1, further comprising a body having a proximal portion defining a mounting device.

20. A fastener system, comprising:
a fastener having a first joinable part and a second joinable part, the first joinable part and the second joinable part being configured for engagement with one another in an interlocking arrangement; and
a fastener delivery device, including:
a first clamp portion configured for removeable engagement with the first joinable part of the fastener; and
a second clamp portion configured for removeable engagement with the second joinable part of the fastener;
wherein the first clamp portion and the second clamp portion are each pivotable between a first position and a second position such that the first clamp portion and the second clamp portion are substantially parallel with one another when in the second position;
wherein drawing the first clamp portion and the second clamp portion towards one another when the first clamp portion and the second clamp portion are substantially parallel causes the first joinable part to engage the second joinable part in the interlocking arrangement.

21. The fastener system of claim 20, further comprising a first leg associated with the first clamp portion and a second leg associated with the second clamp portion, the first clamp portion being pivotable between the first position and the second position along a first face of the first leg and the second clamp portion being pivotable between the first position and the second position along a second face of the second leg.

22. The fastener system of claim 21, where the first leg and the second leg are bifurcations of a distal portion of a body of the fastener delivery device.

23. The fastener system of claim 20, the first joinable part and the second joinable part of the fastener each including a disc body and an opposing face having a plurality of pins interposed between a plurality of receiving apertures.

24. The fastener system of claim 23, wherein the disc body of the first joinable part or the second joinable part includes a disc aperture configured to receive a cylindrical structure and wherein the plurality of pins of the first joinable part or the second joinable part is operable to engage an everted portion of the cylindrical structure.

25. The fastener system of claim 20, the fastener delivery device including a body having a distal portion.

26. The fastener system of claim 25, the body of the fastener delivery device further including an intermediate portion defining one or more curves.

27. The fastener system of claim 20, further including a mounting device in association with the fastener delivery device.

28. A method, comprising:
providing a fastener system, including:
a fastener having a first joinable part and a second joinable part; and
a fastener delivery device configured for removeable engagement with the fastener, the fastener delivery device including a first clamp portion and the first joinable part of the fastener and a second clamp portion and the second joinable part of the fastener;
engaging a first everted portion of a first cylindrical structure with the first joinable part of the fastener and engaging a second everted portion of a second cylindrical structure with the second joinable part of the fastener;
pivoting the first clamp portion and the second clamp portion such that the first clamp portion and the second clamp portion are substantially parallel with one another; and
drawing the first clamp portion and the second clamp portion towards one another such that the first joinable part engages the second joinable part in an interlocking arrangement.

29. The method of claim 28, the first clamp portion being removably engaged with the first joinable part of the fastener and the second clamp portion being removably engaged with the second joinable part of the fastener, the method further comprising:
decoupling the first clamp portion from the first joinable part and decoupling the second clamp portion from the second joinable part.

30. The method of claim 28, the first joinable part and the second joinable part of the fastener each including a disc body defining a disc aperture and an opposing face having a plurality of pins interposed between a plurality of receiving apertures, the method further comprising:
communicating the first cylindrical structure through the disc aperture of the first joinable part and communicating the second cylindrical structure through the disc aperture of the second joinable part; and
everting the first cylindrical structure onto the plurality of pins along the opposing face of the first joinable part and everting the second cylindrical structure onto the plurality of pins along the opposing face of the second joinable part.

31. The method of claim 28, the fastener delivery device including a locking mechanism in operative association with the first clamp portion and the second clamp portion, the method further comprising:
engaging the locking mechanism when the first clamp portion and the second clamp portion are substantially parallel.

32. The method of claim 28, further comprising:
mounting the fastener delivery device to a mounting device.

33. The method of claim 28, the fastener delivery device including a body having an intermediate portion, the method further comprising:
shaping the intermediate portion of the body of the fastener delivery device prior to introduction of the fastener delivery device into an operative field.

34. The method of claim 28, the fastener delivery device including a first leg associated with the first clamp portion and a second leg associated with the second clamp portion, the first clamp portion being pivotable between a first position and a second position along a first face of the first leg and the second clamp portion being pivotable between the first position and the second position along a second face of the second leg, the method further comprising:
pivoting the first clamp portion along the first face of the first leg to the second position and the second clamp portion along the second face of the second leg to the second position such that the first clamp portion and the second clamp portion are substantially parallel with one another; and
drawing the first leg and the second leg towards one another such that the first joinable part engages the second joinable part in the interlocking arrangement.

35. The method of claim 28, wherein the first clamp portion includes a first base and a first receptacle slidably mounted along the first base and configured to receive the first joinable part, the first base being pivotable about a hinge between a first position and a second position, wherein the second clamp portion includes a second base and a second receptacle slidably mounted along the second base and configured to receive the second joinable part, the second base being pivotable about the hinge between the first position and the second position, wherein the method further comprises:
pivoting the first base and the second base about the hinge to the second position such that the first receptacle and the second receptacle are substantially parallel with one another; and
sliding the first receptacle and the second receptacle towards one another such that the first joinable part engages the second joinable part in the interlocking arrangement.
